(19) United States
(12) Patent Application Publication
(10) Pub. No.: US 2009/0131279 A1
(43) Pub. Date: May 21, 2009

(54) NUCLEIC ACID FLUORESCENT STAINS

(75) Inventors: Alexander Rueck, Buchs (CH); Bernhard Schoenenberger, Azmoos (CH)

Correspondence Address: POLSINELLI SHUGART PC, 700 W. 47TH STREET, SUITE 1000, KANSAS CITY, MO, 64112-1802, US

(73) Assignee: SIGMA ALDRICH COMPANY, St. Louis, MO (US)

(21) Appl. No.: 11/940,879

(22) Filed: Nov. 15, 2007

(51) Int. Cl. C40B 50/06; C07D 215/00; C07H 21/00; G01N 31/00

(52) U.S. Cl. 506/26; 546/165; 536/22.1; 436/17

(57) ABSTRACT

The present invention provides fluorescent dye compounds and methods of using the compounds for the staining of nucleic acids. In particular, the dye compounds comprise heterocyclic molecules with hydroxy alkyl and aromatic substituents, and the dye compounds form highly fluorescent complexes upon nucleic acid binding.

### FIELD OF THE INVENTION

The present invention relates to fluorescent dye compounds that non-covalently bind to nucleic acids.

### BACKGROUND OF THE INVENTION

In many areas of life science research, the ability to detect or quantify nucleic acids in pure solutions or in biological samples is critical. In general, the detection methodology should be fast, sensitive, and selective. Some fluorescent nucleic acid stains are particularly sensitive because the fluorescence of the dye increases several orders of magnitude upon binding to DNA. An early fluorescent nucleic acid stain was Thiazole Orange, an unsymmetrical cyanine dye. Over the years, modifications to the heterocyclic moieties of the unsymmetrical cyanine dye molecule have led to the development of improved dyes, i.e., they bind the nucleic acid more tightly, have increased water solubility, and so forth.

Despite these improvements, there is still a need for highly fluorescent nucleic acid stains. In particular, there is a need for nucleic acid stains that have low intrinsic fluorescence but form highly fluorescent complexes upon nucleic acid binding. Such nucleic acid stains would be useful for the detection of nucleic acids on a solid support, such as an electrophoresis gel, in which nucleic acid detection depends largely upon a high signal to noise ratio. Furthermore, the spectral properties of such highly fluorescent nucleic acid stains should be such that these stains can be detected with commonly used detection devices.

### SUMMARY OF THE INVENTION

Among the various aspects of the invention, therefore, are nucleic acid stains that form fluorescent complexes upon nucleic acid binding. Furthermore, these nucleic acid complexes can be detected over a broad range of fluorescence wavelengths, such that they may be detected with a variety of detection devices.

Briefly, accordingly, one aspect of the present invention encompasses a compound comprising Formula (I):

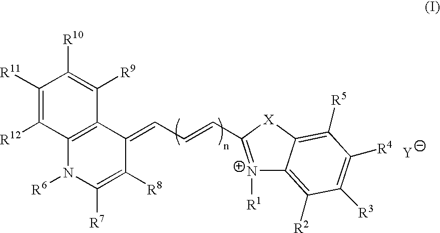

wherein:
R¹ is {—}CH₂(R¹³)ₘOH;
R², R³, R⁴, R⁵, R⁶, R⁸, R⁹, R¹⁰, R¹¹, and R¹² are independently selected from the group consisting of hydrogen, halogen, hydrocarbyl, and substituted hydrocarbyl; provided that any two adjacent substituents may form an aromatic ring or heteroaromatic ring;
R⁷ is a moiety comprising an aromatic ring or a heteroaromatic ring;
R¹³ is selected from the group consisting of hydrocarbyl and substituted hydrocarbyl;
X is a heteroatom;
Y⁻ is a counteranion;
m is an integer from 0 to 10; and
n is an integer from 0 to 5.

Another aspect of the invention encompasses a complex comprising a nucleic acid non-covalently bound to a compound comprising Formula (I), as defined above.

In still another aspect, the invention provides a method for staining a nucleic acid. The method comprises contacting the nucleic acid with a compound to form at least one non-covalently bound compound-nucleic acid complex that produces a detectable fluorescent signal. The compound comprises Formula (I), as defined above.

Other aspects and features of the invention will be in part apparent and in part pointed out hereinafter

### DESCRIPTION OF THE FIGURES

FIG. 1 presents DNA stained after electrophoresis with either compound 6 (SL-2791) (bottom gel images) or SYBR® Green 1 (SG1; Invitrogen Corp., Carlsbad, Calif.) (top gel images). The left lane of each gel contained a total of 500 ng of DNA and the right lane of each gel contained a total of 100 ng of DNA. (A) Imaged with a UV transilluminator and a CCD camera using a 590 nm emission filter. (B) Imaged with a blue light transilluminator and CCD camera using a 590 nm emission filter. (C) Imaged with a laser scanner system using an excitation/emission filter set of 473/520 nm. (D) Imaged with a laser scanner system using an excitation/emission filter set of 532/580 nm.

FIG. 2 presents DNA stained during electrophoresis with either compound 7 (SL-2833) (bottom gel images) or ethidium bromide (EtBr) (top gel images). The left and right lanes of each gel contained a total of 200 ng of DNA and 20 ng of DNA, respectively. (A) Imaged with a UV transilluminator and a CCD camera using a 535 nm emission filter. (B) Imaged with a UV transilluminator and CCD camera using a 590 nm emission filter. (C) Imaged with a laser scanner system using a 473/520 nm filter set. (D) Imaged with a laser scanner system using a 532/580 nm filter set.

FIG. 3 presents DNA stained after electrophoresis with either compound 7 (SL-2833) (top gel images) or compound 8 (SL-2834) (bottom gel images). The left and right lanes of each gel contained a total of 200 ng of DNA and 20 ng of DNA, respectively. (A) Imaged with a UV transilluminator and a CCD camera using a 535 nm emission filter. (B) Imaged with a UV transilluminator and CCD camera using a 590 nm emission filter. (C) Imaged with a blue light transilluminator and CCD camera using a 590 nm emission filter. (D) Imaged with a laser scanner system using a 473/520 nm filter set. (E) Imaged with a laser scanner system using a 532/580 nm filter set.

FIG. 4 presents DNA and RNA stained after electrophoresis with compound 9 (SL-2845), SYBR® Green 1 (SG1), or SYBR® Green 2 (SG2; Invitrogen Corp). The left and right lanes of each gel contained a total of 200 ng of DNA and 1 μg of RNA, respectively. (A) Imaged with a laser scanner system using a 532/580 nm filter set. (B) (D) (F) Imaged with a UV transilluminator and CCD camera using a 590 nm emission filter. (C) (E) Imaged with a laser scanner system using a 473/520 nm filter set.

FIG. 5 illustrates the lower limit of detection of various stains. The left and right lanes of each gel contained a total of 200 ng of DNA and 20 ng of DNA, respectively. DNA was stained with EtBr during electrophoresis or stained with the other compounds after electrophoresis. The gels were imaged with a UV transilluminator and CCD camera using a 590 nm emission filter. The lowest amount of DNA per band detected with each stain is indicated.

FIG. 6 illustrates the digestion of stained DNA. (A) 0.8% agarose gel in which the DNA was stained with compound 6 (SL-2791) and imaged with a laser scanner system. The 6557 bp band was excised over a blue light transilluminator, the DNA was eluted from the agarose and digested. (B) 1.2% agarose gel containing undigested (left lane) and digested (right lane) eluted DNA, stained with 9 (SL-2845), and imaged as in (A).

FIG. 7 illustrates staining of DNA in solution. Arbitrary units (a.u.) of fluorescence are plotted as a function of DNA concentration. (A) presents the fluorescence values for DNA concentrations ranging from 0 to 10 μg/ml. (B) presents an enlarged view of linear portion of the plot (i.e., from 0 to 2 μg/ml). (C) presents an enlarged view of portion of the plot from 0 to 0.5 μg/ml, illustrating that low levels of DNA are detected.

FIG. 8 presents the excitation and emission spectra of compound 9 (SL-2845) in the presence of DNA. Arbitrary units (a.u.) of fluorescence are plotted as a function of wavelength (nm). Excitation spectrum is shown in black and emission spectrum is shown in gray.

### DETAILED DESCRIPTION OF THE INVENTION

Nucleic acid stains have been developed that form highly fluorescent nucleic acid complexes that can be detected over a broad range of fluorescence wavelengths. In particular, the nucleic acid stains are unsymmetrical cyanine dye molecules with 1) a benzthiazole, a benzoxazole, or a benzazole moiety comprising a hydroxy alkyl substituent and 2) a quinoline moiety comprising an aromatic substituent. In general, these substituents on the heterocyclic portions of the dye compound stabilize and increase interactions between the nucleic acid and the dye compound and shift the spectral profile of the bound dye compound to longer wavelengths as compared to commonly used nucleic acid stains.

### I. Fluorescent Dye Compounds

a. Chemical Structures

One aspect of the invention provides fluorescent cyanine dye compounds comprising a hydroxy alkyl substituted benzthiazole moiety linked to an aromatic substituted quinoline moiety. In one embodiment of the invention, the dye compound comprises Formula (I):

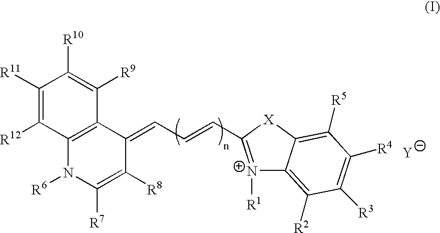

wherein:
R¹ is {—}CH₂(R¹³)ₘOH;
R², R³, R⁴, R⁵, R⁶, R⁸, R⁹, R¹⁰, R¹¹, and R¹² are independently selected from the group consisting of hydrogen, halogen, hydrocarbyl, and substituted hydrocarbyl; provided that any two adjacent substituents may form an aromatic ring or heteroaromatic ring;
R⁷ is a moiety comprising an aromatic ring or a heteroaromatic ring;
R¹³ is selected from the group consisting of hydrocarbyl and substituted hydrocarbyl;
X is a heteroatom;
Y⁻ is a counteranion;
m is an integer from 0 to 10; and
n is an integer from 0 to 5.

In preferred embodiments for compounds having Formula (I), R¹ is {—}CH₂(CH₂)ₘOH, m is from 0 to 5, X is a sulfur atom, and n is from 0 to 3. In exemplary embodiments for compounds having Formula (I), R¹ is {—}CH₂(CH₂)ₘOH, m is from 0 to 5, n is from 0 to 3, R⁷ is a phenyl ring, X is a sulfur atom, and Y⁻ is a perchlorate ion (ClO₄—) or an iodine ion (I—).

In another embodiment, the fluorescent dye compound comprises Formula (II):

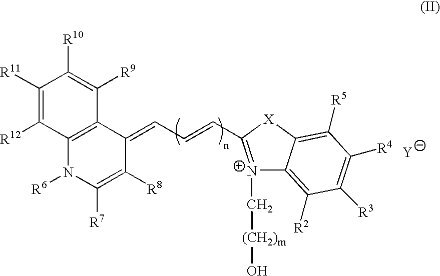

wherein:
R², R³, R⁴, R⁵, R⁶, R⁸, R⁹, R¹⁰, R¹¹, and R¹² are independently selected from the group consisting of hydrogen, halogen, alkyl, alkenyl, and alkoxy; provided that any two adjacent substituents may form an aromatic ring or heteroaromatic ring;
R⁷ is a moiety comprising an aromatic ring or a heteroaromatic ring;
X is selected from the group consisting of a sulfur atom, an oxygen atom, and {—}C(CH₃)₂;
Y⁻ is a counteranion;
m is an integer from 0 to 10; and
n is an integer from 0 to 5.

In preferred embodiments for compounds having Formula (II), X is a sulfur atom, R⁷ is a phenyl ring, m is from 0 to 5, and n is from 0 to 3. In even more preferred embodiments for compounds having Formula (II), R², R³, R⁴, R⁵, R⁸, R⁹, R¹¹, and R¹² are hydrogen, R⁶ and R¹⁰ are independently selected from the group consisting of a hydrogen atom and a methyl group, X is a sulfur atom, R⁷ is a phenyl ring, m is from 0 to 5, and n is from 0 to 3.

In yet another embodiment, the dye compound of the invention comprises Formula (III):

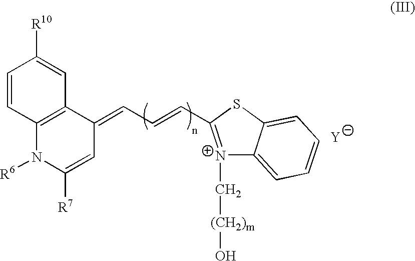

wherein:
R⁶ is selected from the group consisting of a hydrogen atom and a methyl group;
R⁷ is a moiety comprising an aromatic ring or a heteroaromatic ring;
R¹⁰ is selected from the group consisting of a hydrogen atom and a methyl group;
Y⁻ is a counteranion;
m is an integer from 0 to 5; and
n is an integer from 0 to 3.

In preferred embodiments for compounds having Formula (II), R⁷ is a phenyl group. Table A lists exemplary compounds having Formula (III).

TABLE A
Exemplary Formula (III) Compounds.

| R⁶ | R⁷ | R¹⁰ | m | n |
|---|---|---|---|---|
| H | phenyl | H | 0 | 0 |
| methyl | phenyl | H | 0 | 0 |
| H | phenyl | methyl | 0 | 0 |
| methyl | phenyl | methyl | 0 | 0 |
| H | phenyl | H | 1 | 0 |
| methyl | phenyl | H | 1 | 0 |
| H | phenyl | methyl | 1 | 0 |
| methyl | phenyl | methyl | 1 | 0 |
| H | phenyl | H | 2 | 0 |
| methyl | phenyl | H | 2 | 0 |
| H | phenyl | methyl | 2 | 0 |
| methyl | phenyl | methyl | 2 | 0 |
| H | phenyl | H | 3 | 0 |
| methyl | phenyl | H | 3 | 0 |
| H | phenyl | methyl | 3 | 0 |
| methyl | phenyl | methyl | 3 | 0 |
| H | phenyl | H | 4 | 0 |
| methyl | phenyl | H | 4 | 0 |
| H | phenyl | methyl | 4 | 0 |
| methyl | phenyl | methyl | 4 | 0 |
| H | phenyl | H | 5 | 0 |
| methyl | phenyl | H | 5 | 0 |
| H | phenyl | methyl | 5 | 0 |
| methyl | phenyl | methyl | 5 | 0 |
| H | phenyl | H | 0 | 1 |
| methyl | phenyl | H | 0 | 1 |
| H | phenyl | methyl | 0 | 1 |
| methyl | phenyl | methyl | 0 | 1 |
| H | phenyl | H | 1 | 1 |
| methyl | phenyl | H | 1 | 1 |
| H | phenyl | methyl | 1 | 1 |
| methyl | phenyl | methyl | 1 | 1 |
| H | phenyl | H | 2 | 1 |
| methyl | phenyl | H | 2 | 1 |
| H | phenyl | methyl | 2 | 1 |
| methyl | phenyl | methyl | 2 | 1 |
| H | phenyl | H | 3 | 1 |
| methyl | phenyl | H | 3 | 1 |
| H | phenyl | methyl | 3 | 1 |
| methyl | phenyl | methyl | 3 | 1 |
| H | phenyl | H | 4 | 1 |
| methyl | phenyl | H | 4 | 1 |
| H | phenyl | methyl | 4 | 1 |
| methyl | phenyl | methyl | 4 | 1 |
| H | phenyl | H | 5 | 1 |
| methyl | phenyl | H | 5 | 1 |
| H | phenyl | methyl | 5 | 1 |
| methyl | phenyl | methyl | 5 | 1 |
| H | phenyl | H | 0 | 2 |
| methyl | phenyl | H | 0 | 2 |
| H | phenyl | methyl | 0 | 2 |
| methyl | phenyl | methyl | 0 | 2 |
| H | phenyl | H | 1 | 2 |
| methyl | phenyl | H | 1 | 2 |
| H | phenyl | methyl | 1 | 2 |
| methyl | phenyl | methyl | 1 | 2 |
| H | phenyl | H | 2 | 2 |
| methyl | phenyl | H | 2 | 2 |
| H | phenyl | methyl | 2 | 2 |
| methyl | phenyl | methyl | 2 | 2 |
| H | phenyl | H | 3 | 2 |
| methyl | phenyl | H | 3 | 2 |
| H | phenyl | methyl | 3 | 2 |
| methyl | phenyl | methyl | 3 | 2 |
| H | phenyl | H | 4 | 2 |
| methyl | phenyl | H | 4 | 2 |
| H | phenyl | methyl | 4 | 2 |
| methyl | phenyl | methyl | 4 | 2 |
| H | phenyl | H | 5 | 2 |
| methyl | phenyl | H | 5 | 2 |
| H | phenyl | methyl | 5 | 2 |
| methyl | phenyl | methyl | 5 | 2 |
| H | phenyl | H | 0 | 3 |
| methyl | phenyl | H | 0 | 3 |
| H | phenyl | methyl | 0 | 3 |
| methyl | phenyl | methyl | 0 | 3 |
| H | phenyl | H | 1 | 3 |
| methyl | phenyl | H | 1 | 3 |
| H | phenyl | methyl | 1 | 3 |
| methyl | phenyl | methyl | 1 | 3 |
| H | phenyl | H | 2 | 3 |
| methyl | phenyl | H | 2 | 3 |
| H | phenyl | methyl | 2 | 3 |
| methyl | phenyl | methyl | 2 | 3 |
| H | phenyl | H | 3 | 3 |
| methyl | phenyl | H | 3 | 3 |
| H | phenyl | methyl | 3 | 3 |
| methyl | phenyl | methyl | 3 | 3 |
| H | phenyl | H | 4 | 3 |
| methyl | phenyl | H | 4 | 3 |
| H | phenyl | methyl | 4 | 3 |
| methyl | phenyl | methyl | 4 | 3 |
| H | phenyl | H | 5 | 3 |
| methyl | phenyl | H | 5 | 3 |
| H | phenyl | methyl | 5 | 3 |
| methyl | phenyl | methyl | 5 | 3 |

In exemplary embodiments for compounds having Formula (III), R⁶ is a methyl group, R⁷ is a phenyl group, n is 0, and Y⁻ is selected from the group consisting of ClO₄— and I—. Exemplary dye compounds include compounds 6, 7, 8, 9, 10, and 11, which are presented below in Table B.

TABLE B
Exemplary Dye Compounds

| Compound | |
|---|---|
| Number or Name | Structure |
| 6 (SL-2791) | 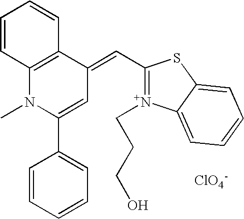 |
| 7 (SL-2822) | 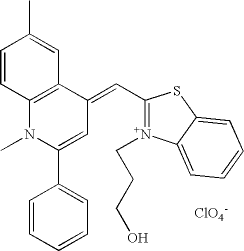 |
| 8 (SL-2834) | 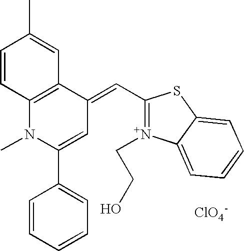 |
| 9 (SL-2845) | 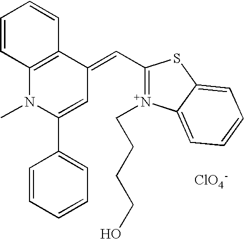 |
| 10 (SL-2828) | 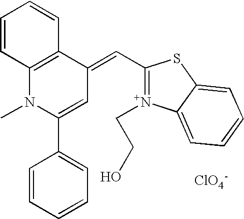 |
| 11 (SL-2792) | 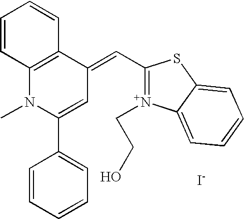 |

b. Properties of the Dye Compounds

The dye compounds of the invention specifically bind nucleic acids, with moderate to high affinity. The nucleic acid may be DNA, RNA, or a combination thereof.

Without being bound by any particular theory, it is believed that the hydroxy alkyl substituent on the nitrogen atom of the benzthiazole (or benzoxazole or benzazole) moiety of the dye compound enhances binding to a nucleic acid via the formation of additional hydrogen bonds and Van der Waals interactions. As a consequence, the dye compound appears to be fixed in a more rigid position in a groove of the nucleic acid, resulting in an increase of the binding constant of the dye compound-nucleic acid complex. In general, an increased binding constant means that an increased number of dye compound molecules are able to bind to the nucleic acid at a given concentration of dye compound and nucleic acid. Accordingly, an increased number of dye compound molecules fixed on the surface of the nucleic acid generally leads to an increase in fluorescence signal.

In general, it appears that the hydroxy alkyl substituent on the benzthiazole moiety of the dye compound does not significantly alter the spectral properties of the dye compounds. Without being bound by any particular theory, it is believed, however, that the aromatic substituent on position 2 of the quinoline moiety of the dye compound shifts the absorption and fluorescence emission maxima of the dye compound to longer wavelengths relative to those of commonly nucleic acid stains having the same basic molecular structure. As shown in Examples 2 and 3, nucleic acid complexes comprising a dye compound of the invention were also detected upon excitation at 532 nm using a 580 emission filter, whereas nucleic acid complexes comprising SYBR® Green 1 were not detected and only high levels of complexes comprising ethidium bromide were detected.

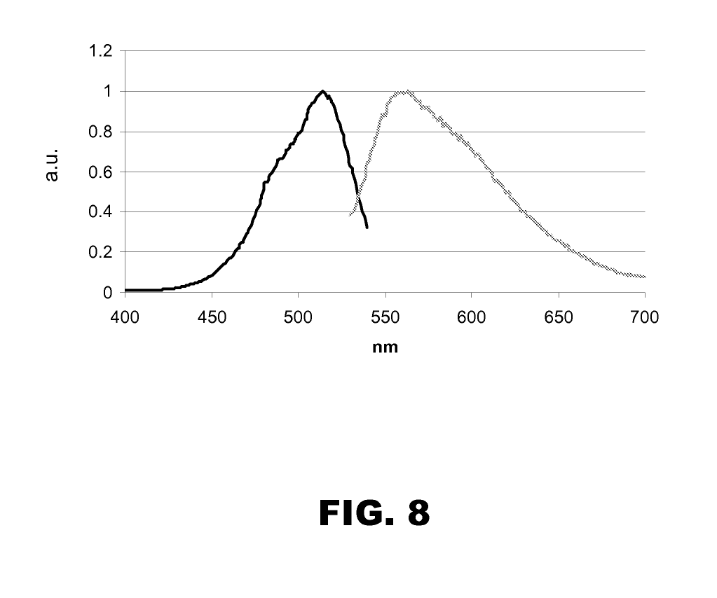

The dye compounds of the invention generally have absorption maxima ranging from about 500 nm to about 580 nm (e.g., see FIG. 8 for an example). In one embodiment, the absorption maximum of the dye compound is about 500 nm. In another embodiment, the absorption maximum of the dye compound is about 510 nm. In yet another embodiment, the absorption maximum of the dye compound is about 520 nm. In still another embodiment, the absorption maximum of the dye compound is about 530 nm. In another embodiment, the absorption maximum of the dye compound is about 540 nm. In an alternate embodiment, the absorption maximum of the dye compound is about 550 nm. In another alternate embodiment, the absorption maximum of the dye compound is about 560 nm. In another alternate embodiment, the absorption maximum of the dye compound is about 570 nm. In still another alternate embodiment, the absorption maximum of the dye compound is about 580 nm.

Furthermore, the dye compounds of the invention may have additional absorption peaks in the ultraviolet range, which allows then to be excited by a wide range of wavelengths. In one embodiment, a dye compound of the invention may be excited by light of about 254 nm. In another embodiment, a dye compound of the invention may be excited by light of about 300 nm. In yet another embodiment, a dye compound of the invention may be excited by light of about 450 nm. In still another embodiment, a dye compound complex of the invention may be excited by light of about 473 nm. In an alternate embodiment, a dye compound complex of the invention may be excited by light of about 490 nm. In another alternate embodiment, a dye compound of the invention may be excited by light of about 532 nm.

The emission maxima of the dye compounds of the invention generally range from about 520 nm to about 600 nm (e.g., see FIG. 8 for an example). In one embodiment, the emission maximum of the dye compound may be about 520 nm. In another embodiment, the emission maximum of the dye compound may be about 530 nm. In yet another embodiment, the emission maximum of the dye compound may be about 540 nm. In still another embodiment, the emission maximum of the dye compound may be about 550 nm. In an alternate embodiment, the emission maximum of the dye compound may be about 560 nm. In another alternate embodiment, the emission maximum of the dye compound may be about 570 nm. In still another embodiment, the emission maximum of the dye compound may be about 580 nm. In another alternate embodiment, the emission maximum of the dye compound may be about 590 nm. In still another alternate embodiment, the emission maximum of the dye compound may be about 600 nm.

### II. Fluorescent Dye Compound-Nucleic Acid Complexes

Another aspect of the invention encompasses a complex comprising at least one fluorescent dye compound non-covalently bound to a nucleic acid. The fluorescent dye compounds were described above in Section I. The nucleic acid may be DNA, RNA, or a combination thereof. The DNA may be single-, double-, triple-, or quadruple-stranded, and the RNA may be single- or double-stranded. Alternatively, the nucleic acid may be a branched DNA or RNA molecule. In general, the nucleic acid will be at least 10 nucleotides in length. The nucleic acid may be a naturally occurring molecule, or a synthetic molecule. The nucleic acid may comprise standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos. The nucleotides of the nucleic acid may be linked by phosphodiester, phosphothioate, phosphoramidite, or phosphorodiamidate bonds.

When a fluorescent dye compound binds to a nucleic acid, it exhibits an enhancement of the fluorescent signal. Stated another way, the fluorescent dye compound has low intrinsic fluorescence, but its fluorescence increases upon binding to a nucleic acid. In general, the fluorescence enhancement of a dye compound increases at least several hundred-fold upon binding to a nucleic acid. In one embodiment, the fluorescence enhancement of the bound dye molecule may be about 50-fold. In another embodiment, the fluorescence enhancement of the bound dye molecule may be about 100-fold. In yet another embodiment, the fluorescence enhancement of the bound dye molecule may be about 300-fold. In still another embodiment, the fluorescence enhancement of the bound dye molecule may be about 500-fold. In an alternate embodiment, the fluorescence enhancement of the bound dye molecule may be about 700-fold. In another alternate embodiment, the fluorescence enhancement of the bound dye molecule may be about 1000-fold. In yet another embodiment, the fluorescence enhancement of the bound dye molecule may be about 3000-fold.

As detailed above, the hydroxy alkyl substituent on the benzthiazole (or benzoxazole or benzazole) moiety of the dye compound increases hydrogen bonding and other interactions between the dye compound and the nucleic acid. Further it appears that the dye compounds bind preferentially, but not exclusively, to nucleic acid grooves. Accordingly, the dye molecules bind DNA more tightly than RNA. Thus, complexes comprising DNA exhibit increased fluorescence relative to those comprising RNA, as shown in Example 5.

The high number of dye molecules bound to a nucleic acid not only increases the fluorescence signal, but also permits detection of lower quantities of nucleic acids relative to commonly used nucleic acid stains, as demonstrated in Examples 2 and 3. The amount of nucleic acid detected by the dye compounds of the invention can and will vary, depending upon a variety of factors, including the detection means. In one embodiment, the dye compound may detect about 10 pg of DNA. In another embodiment, the dye compound may detect about 50 pg of DNA. In an alternate embodiment, the dye compound may detect about 250 pg of DNA. In still another embodiment, the dye compound may detect about 1 ng of DNA. In another alternate embodiment, the dye compound may detect about 5 ng of DNA. In still another alternate embodiment, the dye compound may detect about 0.5 μg of RNA. In another embodiment, the dye compound may detect about 1 μg of RNA. In still another embodiment, the dye compound may detect about 5 μg of RNA.

### III. Methods of Staining Nucleic Acids

A further aspect of the invention provides methods for staining nucleic acids. The method comprises contacting the nucleic acid with a dye compound of the invention to form at least one dye compound-nucleic acid complex, whereby the dye compound-nucleic acid complex produces a detectable fluorescent signal. The dye compounds and dye compound-nucleic acid complexes were detailed above in Sections I and II, respectively.

The source of the nucleic acid can and will vary. In one embodiment, the nucleic acid may be isolated or purified from a natural source or a chemical synthesis reaction. In another embodiment, the nucleic acid may be part of an enzymatic or biochemical reaction. In an alternate embodiment, the nucleic acid may be unpurified in that it is provided in a cell homogenate or an extract of a cell. The cell may be eukaryotic or prokaryotic. In still another embodiment, the nucleic acid may be provided in a eukaryotic cell, an organelle, a chromosome, a prokaryotic cell, a microorganism, or a virus.

In general, the nucleic acid is contacted with the dye molecule under conditions that permit the formation of dye molecule-nucleic acid complexes. The concentration of the dye molecule and the duration of contact time can and will vary upon the application.

a. Solid Support Applications

In one embodiment, the dye molecule-nucleic acid complexes may be detected on a solid support. The solid support may be an electrophoretic matrix. Non-limiting examples of suitable electrophoretic matrices include horizontal gels, vertical gels, capillary gels, agarose gels, polyacrylamide gels, polymer gels, and silica gel capillaries. In a preferred embodiment, the solid support may be an agarose gel, as detailed in Examples 2-6. The agarose gel containing the electrophoretically separated nucleic acids may be immersed in a solution comprising a dye compound of the invention. Typically, the concentration of the dye compound may range from about 0.1 μM to about 10 μM, or more preferably from about 0.5 μM to about 2 μM. The dye solution may optionally comprise a buffer, such as TBE, TAE, phosphate, Tris, or PBS. The length of time of contact with the dye solution can and will vary, depending on the thickness of the gel, for example. In general, the staining time may range from about 5 minutes to about 2 hours, or more preferably about 1 hour, at room temperature. The gel may be destained in an aqueous solution, but this step is generally not required. In another preferred embodiment, the nucleic acid may be contacted with the dye prior to being loaded onto the agarose gel. The concentration of the dye molecule is generally the same as that used to stain a gel after electrophoresis. In general, gels stained with the dye compounds of the invention will have high signal to noise ratios because of the low intrinsic fluorescence of the dye compounds and the enhanced fluorescence of the dye compound-nucleic acid complexes.

In an alternative of this embodiment, the stained nucleic acid complex may be extracted from the agarose gel and subjected to an enzymatic reaction. The binding of the dye compound to the nucleic acid generally does not affect the ability of an enzyme to catalyze a reaction in which the nucleic acid is a substrate, as shown in Example 7. The reaction may be catalyzed by a restriction endonuclease, an exonuclease, a DNA polymerase, a DNA ligase, an RNA polymerase, an RNA ligase, and other nucleic acid modifying enzymes.

In another embodiment, the solid support may be a transfer membrane, such as a nitrocellulose or nylon membrane. Typically, dye molecule-nucleic acid complexes are transferred to the membrane from a stained gel. In a further embodiment, the solid support may be a microarray comprising immobilized oligonucleotides or nucleic acids. In general, the target nucleic acid may be contacted with the dye molecule prior to or during exposure to the immobilized oligonucleotides or nucleic acids on the microarray.

The fluorescence of the dye compound-nucleic acid complexes immobilized on or embedded in a solid support may be detected with standard detection devices. A dye compound-nucleic acid complex may be excited by a light source capable of producing light at or near the wavelength of the absorption maximum of the complex. Suitable examples of light sources include ultraviolet epi- and transilluminators, blue light transilluminators, mercury-arc lamps, and lasers. The laser may be a diode laser with either 473 nm or 532 excitation, any other diode laser, a HeCd laser (442 nm excitation), a blue Nd:YAG laser (473 nm excitation), an argon laser (488 nm excitation), a green Nd:YAG laser (532 nm excitation), a green HeNe laser (543 nm excitation), or a Kr laser (568 nm excitation). The fluorescence of the complex may be detected and documented with CCD cameras, video cameras, photographic film, or with instrumentation such as CCD-based imaging systems, laser-based scanning systems, plate readers, laser-based microarray readers, capillary electrophoresis detectors, and the like.

b. Aqueous Applications

In yet another embodiment, the nucleic acid may be contacted with the dye compound in an aqueous solution, as demonstrated in Example 8. Detection of dye compound-nucleic acid complexes in an aqueous solution may be used to determine the presence of a nucleic acid in a sample or the quantity of a nucleic acid in the sample. Furthermore, a dye compound of the invention may be used to quantify the level of a nucleic acid during amplification reactions, such as real-time quantitative PCR (qPCR), ligation-mediated amplifications, real-time strand displacement amplification, rolling circle amplification, multiple-displacement amplification, and other amplification methods (see Demidov and Broude, 2004, DNA Amplifications: Current Technologies and Applications, Horizon Scientific Press, Norwich, U.K., which is incorporated herein by reference). The concentration of the dye compound may range from about 0.1 μM to about 10 μM, and more preferably from about 1 μM to about 2 μM. The dye compound-nucleic acid complexes may be detected with a spectrophotometer, a fluorometer, a laser scanner, a real time PCR machine, a flow cytometer, a quantum counter, and the like.

c. Cellular Applications

In still another embodiment, a cell or fragment thereof comprising the nucleic acid may be contacted with the dye compound. In general, the cell will have permeabilized or compromised cell membranes, such that the dye molecules may readily enter and bind to the nucleic acid. The binding of dye compounds to cell based nucleic acids may be used to distinguish dead cells from live cells (into which the dye molecules are unable to enter). Alternatively, cell based nucleic acid staining may be used to sort cells. The staining of cell-based nucleic acids may also be used to detect the location of the nucleic acid. As an example, the dye compounds of the invention may be used to counterstain the nuclei of cells during immunolocalization studies. The concentration of the dye molecule that is contacted with the cell-based nucleic acid may range from about 0.1 nM to about 50 μM, preferably from about 1 nM to about 10 μM, and more preferably from about 0.5 μM to about 5 μM. The stained nucleic acid complexes may be detected with an epifluorescence microscope, a confocal microscope, a scanning microscope, a flow cytometer, a fluorometer, and a plate reader.

### Definitions

To facilitate understanding of the invention, a number of terms are defined below.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “alkoxy” as used herein denotes an alkyl group linked via an oxygen atom to another moiety.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “counteranion” as used herein denotes a negatively charged group. Suitable counteranions include perchlorate ion (ClO₄—), and a halide ion, such as iodine (I—), chlorine (Cl—), and bromine (Br—).

The terms “halogen” or “halo” as used herein alone or as part of another group refer to chlorine, bromine, fluorine, and iodine.

The term “heteroatom” as used herein refers to atoms other than carbon and hydrogen. Suitable heteroatoms include nitrogen, oxygen, sulfur, phosphorus, boron, chlorine, bromine, and iodine.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters, and ethers.

The term “heteroaromatic” as used herein alone or as part of another group denote optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters, and ethers.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl, and alkynaryl. Unless otherwise indicated, these moieties preferably comprise 1 to 20 carbon atoms.

The term “hydroxy alkyl” as used herein denotes an alkyl group linked to another moiety, the alkyl group having a terminal hydroxyl group.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorous, boron, sulfur, or a halogen atom. These substituents include halogen, carbocycle, aryl, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters, and ethers.

As various changes could be made in the above compounds, complexes, and methods without departing from the scope of the invention, it is intended that all matter contained in the above description and in the examples presented below, shall be interpreted as illustrative and not in a limiting sense.

### EXAMPLES

The following examples illustrate various embodiments of the invention.

### Example 1

### Synthesis of 1-Methyl-4-[(2,3-dihydro-3-(3-hydroxypropyl)benzo-1,3-thiazol-2-yl)-methyliden]-2-phenyl-quinolinium perchlorate (6)

Dyes of the present invention were prepared according to synthetic principles as outlined, for example, by F. Hamer in “The Cyanine Dyes and Related Compounds” (The Chemistry of Heterocyclic Compounds, Vol. 18, A. Weissberger ed., Interscience Publishers, New York, 1964). In brief, a nucleophilic benzazole component was condensed with an electrophillic quinoline moiety resulting in an unsymmetrical monomethincyanine dye.

1-Methyl-4-[(2,3-dihydro-3-(3-hydroxypropyl)benzo-1,3-thiazol-2-yl)-methyliden]-2-phenyl-quinolinium perchlorate (6) was synthesized according to the reaction scheme presented and detailed below.

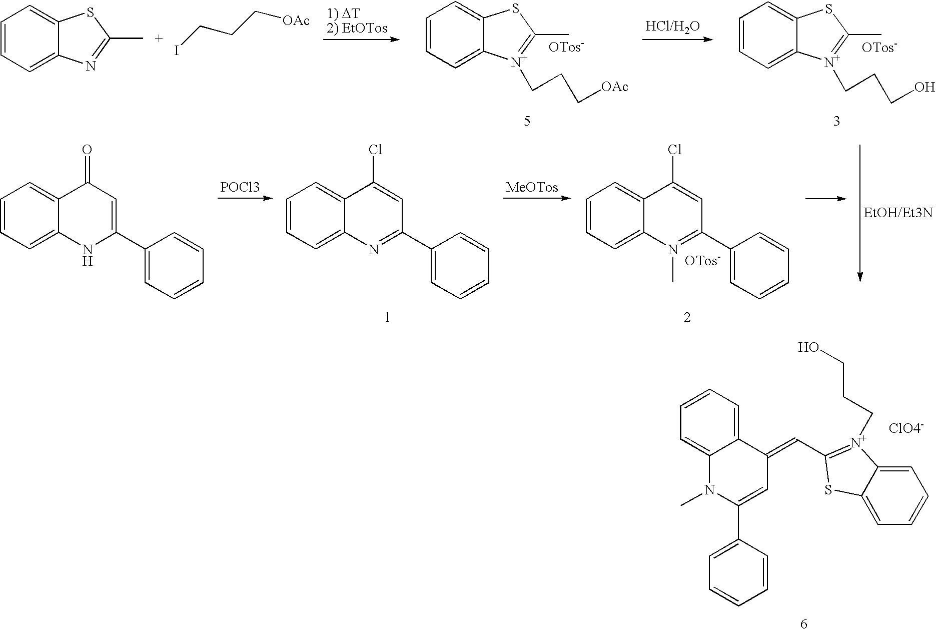

4-Chloro-2-phenyl quinoline (1): 10 g (0.045 mol) of 2-phenyl-4-quinoline was refluxed in 70 ml of phosphorous oxychloride and 0.5 ml of DMF for 40 min. The mixture was evaporated under vacuum to remove the excess phosphorous oxychloride, and the residue was poured into 0.5 kg of ice. The mixture was neutralized to ˜pH 7.5 with conc. aqueous ammonia at 7-10° C. The solid residue was filtered, washed with water and dried to obtain 10.5 g of crude product. The product was suspended in 150 ml of hexane and refluxed until the solubilization was complete. Silica gel was added to the mixture, which was then shaken and the solution was filtered off. The mother solution was evaporated to ⅓ of its original volume. The product was filtered. The yield was 8.14 g (75%). NMR ¹H in CDCl₃: m. 7.52 (3H), t.d. 7.60 (1H, 2 Hz, 9 Hz), t. d. 7.76 (1H, 2 Hz, 9 Hz), s. 7.95 (1H), m. 8.14 (2H), m. 8.19 (2H).

4-Chloro-1-methyl-2-phenyl-quinolinium p-toluenesulfonate (2): 0.72 g (0.003 mol) of 1 and 0.84 g (0.0045 mol) of methyl p-toluenesulfonate were reacted at 125° C. in an oil bath for 5 hours. After cooling, the complex was dissolved in 10 ml of dichloromethane and the mixture was diluted with 50 ml of ether. An oily precipitate gradually formed. The precipitate was filtered, washed with dry ether and dried in a vacuum desiccator under P₂O₅. The yield was 0.93 g (73%).

3-(3-Hydroxypropyl)-2-methyl-benzothiazolium p-toluenesulfonate (3): 5.3 g (36 mmol) of 2-methyl-benzothiazole and 9.8 g (43 mmol) of 3-iodopropyl acetate were mixed and heated for 20 hours at 125-130° C. The solid was triturated with dry acetone, which was then filtered off, and the solid was washed with dry acetone and dry ether. The yield of 3-(3-acetoxypropyl)-2-methyl-benzothiazolium iodide (4) was 11.5 g (85%). 4 was heated at 90° C. with 37 g (0.185 mol) of ethyl p-toluenesulfonate for 40 min until the solid was completely dissolved. The mixture was then heated at 105° C. for 1.3 hours until the ethyl iodide bubbles disappeared. The warm solution was poured into 150 ml of ethyl acetate. After 12 hours, the solid residue was filtered off, and washed with ethyl acetate and dry ether. The yield of 3-(3-acetoxypropyl)-2-methyl-benzothiazolium p-toluenesulfonate (5) was 10.1 g (67% on 2-methyl-benzothiazole). 10.1 g of this salt, 36 ml of water and 7.5 ml of conc. hydrochloric acid were heated at 50° C. for 2 hours. The solution was allowed to stand at room temperature for 12 hours, after which it was evaporated to dryness. The residue was dissolved in 20 ml of water, and activated charcoal was added. The solution was filtered off and evaporated to dryness again. The residue was treated with 25 ml of methanol, which was evaporated off. This operation was repeated twice and the resultant oil was dried in a vacuum desiccator initially over P₂O₅, and then over sodium hydroxide. The viscous product was triturated with acetonitrile to seed the crystal and was recrystallized from acetonitrile. The yield was 5.0 g of (3) (47% on 2-methyl-benzothiazole), m.p. 128-130° C. NMR ¹H in DMSO d⁶: q. 2.03 (2H, 5.5 Hz), s. 2.28 (3H), s. 3.22 (3H), t. 3.52 (2H, 5.5 Hz), t. 4.78 (2H, 5.5 Hz), br.p. ˜4.9 (1H), d. 7.11 (2H, 9.0 Hz), d. 7.46 (2H, 9.0 Hz), t. 7.98 (1H, 8.5 Hz), t.d. 7.89 (1H, 8.5 Hz, 1.3 Hz), d. 8.29 (1H, 9.3 Hz), d. 8.42 (1H, 8.7 Hz).

1-Methyl-4-[(2,3-dihydro-3-(3-hydroxypropyl)benzo-1,3-thiazol-2-yl)-methyliden]-2-phenyl-quinolinium perchlorate (6): 0.43 g (0.001 mol) of 4-chloro-1-methyl-2-phenyl-quinolinium p-toluenesulfonate (2) and 0.38 g (0.001 mol) of 2-methyl-3-(3-hydroxypropyl)benzothiazolium p-toluenesulfonate (3) were mixed in 3 ml of anhydrous alcohol and 0.25 ml (0.18 g, 0.0018 mol) of triethylamine was added. The mixture was refluxed about 1 min. After the solids were completely dissolved, the mixture was allowed to cool and then a solution of 0.5 g of sodium perchlorate in 3 ml of alcohol was added. A solid precipitate appeared and the mixture was diluted with 15 ml of water. The crude product was filtered and recrystallized from 8 ml of acetonitrile. The yield was 0.05 g (10%) of 6, λₘₐₓ 513 nm, ε=7.07′10⁴ M⁻¹cm⁻¹ (methanol). NMR ¹H in DMSO d⁶: t. (3H, 7.5 Hz), br.t. 3.6 (2H), s. 3.91 (3H), t. 4.65 (2H, 7.5 Hz), br.t. 5.05 (1H), t. 7.12 (1H), t. 7.27 (1H), t. 7.40 (1H, 8.5 Hz), t. 7.60 (1H, 9 Hz), m. 7.72 (3H), m. 7.80 (4H), m. 8.04 (2H), d. 8.16 (1H, 9.7 Hz), d. 8.77 (1H, 9 Hz).

Other unsymmetrical monomethincyanine dyes (i.e., compounds 7, 8, 9, 10, and 11) were synthesized using similar reaction strategies.

### Example 2

### Post Electrophoretic Staining of DNA with Compound 6 (SL-2791)

The absorption and emission spectra of the unsymmetrical monomethincyanine dyes of the invention are shifted to longer wavelengths relative to those of commonly used nucleic acid stains. Electrophoretically separated DNA was stained with either a compound of the invention, compound 6 (also called SL-2791), or the nucleic acid stain, SYBR® Green 1 (SG1; Invitrogen Corp.) and imaged using different detection devices.

PstI-digested lambda DNA (Cat. No. D1793; Sigma-Aldrich, St. Louis, Mo.) was resolved on agarose gels in the presence of TBE buffer, pH 8.3. Lanes were loaded with a total of either 500 ng or 100 ng of DNA (the loading buffer contained 0.05% bromophenol blue (w/v), 40% sucrose (w/v), 0.5% SDS (w/v), and 0.1 M EDTA, pH 8). The gels were run at 90 V for about 90 min. After electrophoresis, the gels were submerged in a solution of TBE buffer comprising 2 μM of 6 (SL-2791) or SG1 for 1 hr. After a quick rinse, the gels were imaged with 1) a 300 nm UV transilluminator (Cat. No. T2202; Sigma-Aldrich) or a 450 nm (blue light) transilluminator (Dark Reader™; Clare Chemical Research, Denver, Colo.) and a CCD camera imaging system (Gel Logic 100; Kodak Imaging, Rochester, N.Y.) equipped with a 535 nm or a 590 nm emission filter and 2) a laser scanner fluorescent image analyzer system (FLA-300; Fujifilm, Japan) equipped with excitation/emission filter sets of 473/520 nm or 532/580 nm.

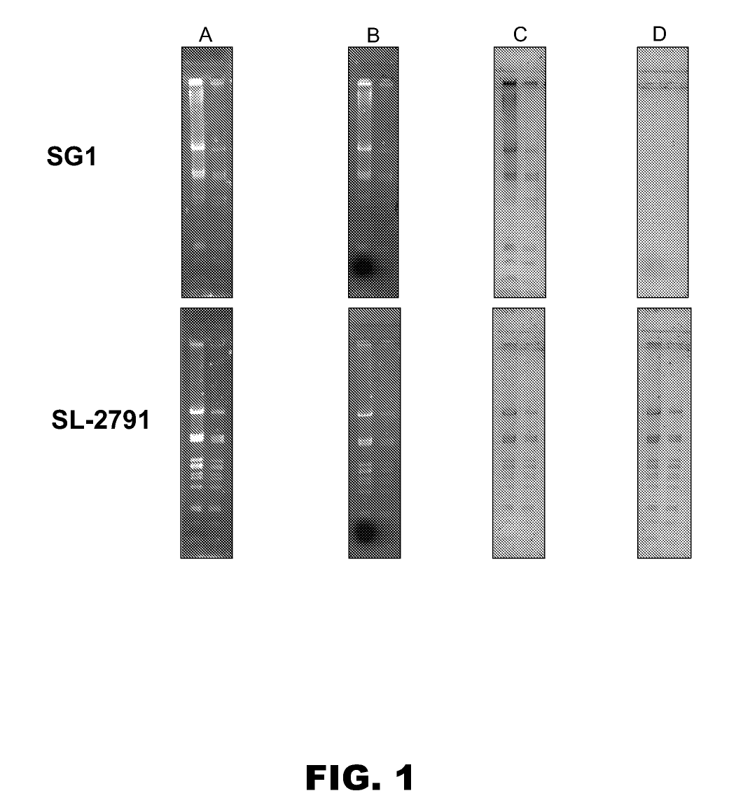

The results are presented in FIG. 1. The bands of DNA stained with 6 (SL-2791) were much brighter than those stained with SG1 under each detection condition. DNA stained with 6 (SL-2791) could be detected at longer wavelengths than DNA stained with SG1. In particular, SG1-stained DNA was not visible with the laser scanner using the 532/580 filter set (see FIG. 1D).

### Example 3

### Prestaining of DNA with Compound 7 (SL-2833)

The ability of another of the new unsymmetrical monomethincyanine dyes, i.e., compound 7 (SL-2833), to stain DNA during electrophoresis (i.e., “prestain”) was compared to that of ethidium bromide (EtBr). For this, 1 μM of 7 (SL-2833) or EtBr was added to the heated agarose before the gel was poured. The gels were loaded with 200 ng/lane and 20 ng/lane of HindIII-digested lambda DNA (Cat. No. D9780; Sigma-Aldrich) and run at 90 V for about 90 min. After electrophoresis, the gels were imaged as described above in Example 2.

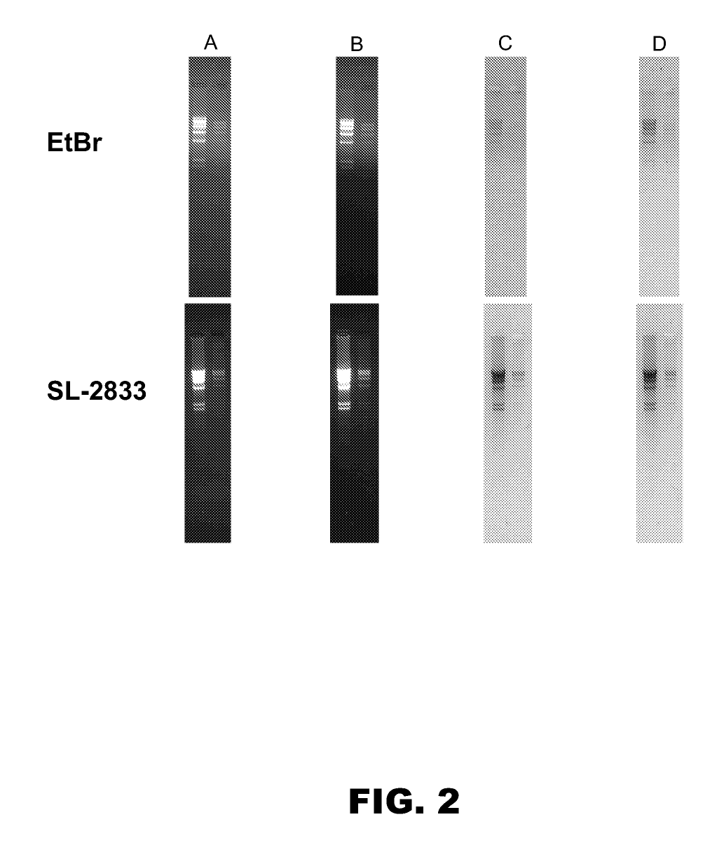

FIG. 2 presents the results. The DNA bands stained with 7 (SL-2833) and imaged with a UV transilluminator displayed increased fluorescence and the top of the gels exhibited lower background fluorescence than those stained with EtBr (FIGS. 2A and B). DNA stained with 7 (SL-2833) was readily imaged at longer wavelengths, whereas low levels of DNA (i.e., 20 ng/lane) stained with EtBr were not detected (FIGS. 2C and D).

### Example 4

### DNA Staining with Compound 7 (SL-2833) or Compound 8 (SL-2834)

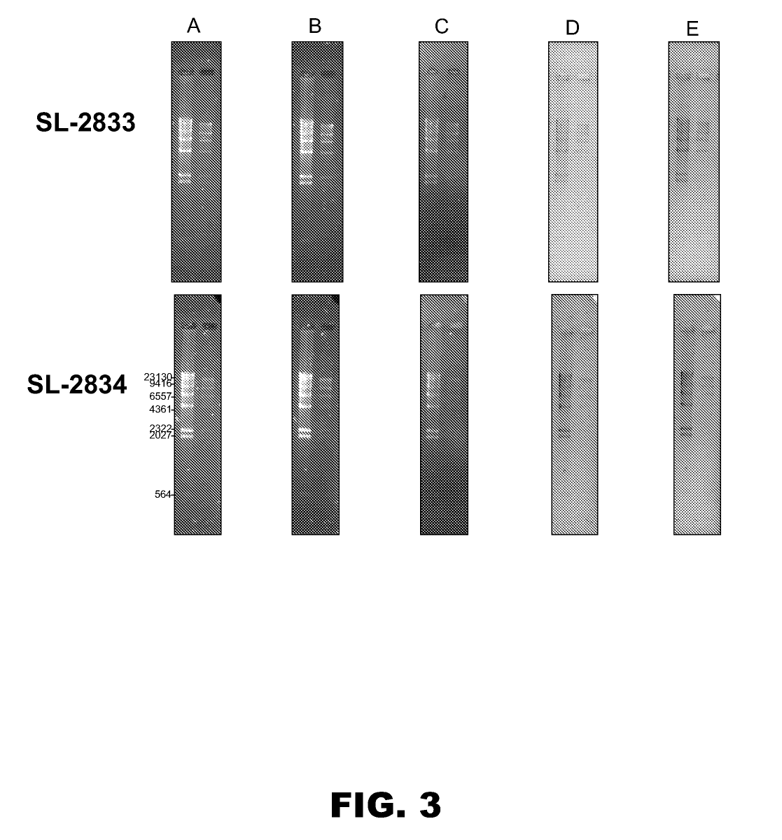

The brightness, sensitivity, and spectral properties of two of the new unsymmetrical monomethincyanine dyes, compound 7 (SL-2833) and compound 8 (SL-2834), were compared by staining gels containing 200 ng/lane and 20 ng/lane of HindIII-digested lambda DNA. The gels were processed and imaged as described above in Example 2. As shown in FIG. 3, both dyes exhibited similar characteristics. For example, DNA stained with either dye exhibited reduced fluorescence when imaged with a blue light transilluminator and a 590 nm filter (FIG. 3C) as compared to a UV transilluminator (FIGS. 3A and B). But DNA stained with either dye was clearly detected using the laser-based long wavelength imaging system (FIGS. 3D and E).

### Example 5

### Staining of DNA and RNA

Gels were loaded with 200 ng/lane of HindIII-digested lambda DNA and 1 μg/lane of 0.2-10 kb RNA markers (Cat. No. R1386; Sigma-Aldrich). The RNA loading buffer contained 62.5% formamide (v/v), 1.14 M formaldehyde, 0.2 mg/ml bromophenol blue, 0.2 mg/ml xylene cyanol, and 1.25× MOPS-EDTA-sodium acetate buffer. After electrophoresis the gels were stained with compound 9 (SL-2845), SG1, or the RNA-specific stain SYBR® Green 2 (SG2; Invitrogen) and imaged as described above in Example 2.

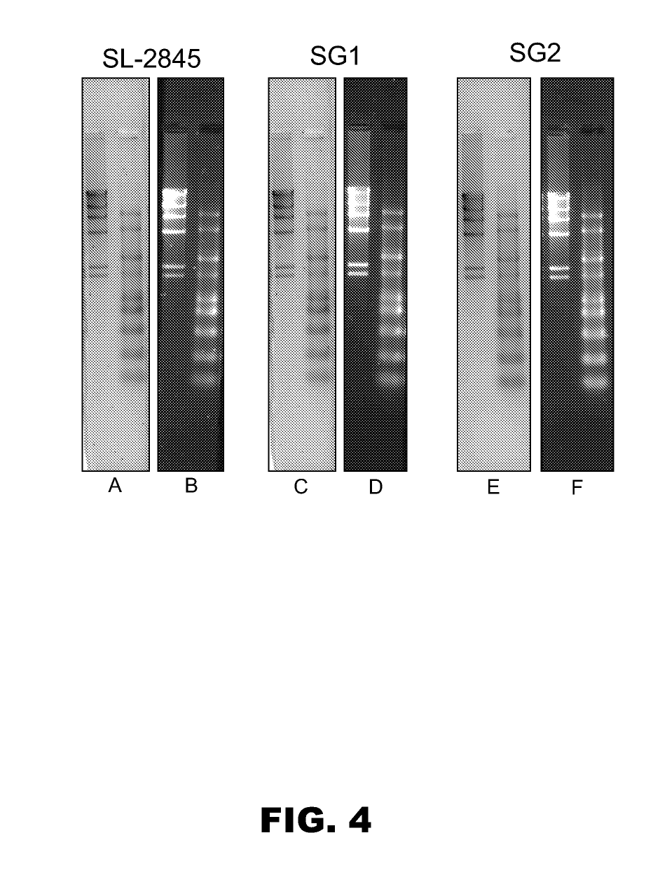

FIG. 4 presents the results. Note, the gel stained with compound 9 (SL-2845) was imaged with the laser scanner system using a 532/580 nm filter set (FIG. 4A), whereas the gels stained with SG1 or SG2 were imaged with the laser scanner using the 473/520 nm filter set (FIGS. 4C and E). Compound 9 (SL-2845) stained DNA more intensely than RNA. These findings indicate that the asymmetric monomethincyanine dyes of the invention have higher affinity for double stranded nucleic acids.

### Example 6

### Determination of the Lower Limit of Detection

HindIII-digested lambda DNA (200 ng/lane and 20 ng/lane) was resolved on agarose gels, as described above. EtBr was added to one gel such that the DNA was stained during electrophoresis; the other gels were stained with compound 6 (SL-2791), 7 (SL-2833), 8 (SL-2834), 9 (SL-2845), or SG1 after electrophoresis. Gels were imaged using a 300 nm UV transilluminator and a CCD camera with a 590 nm emission filter.

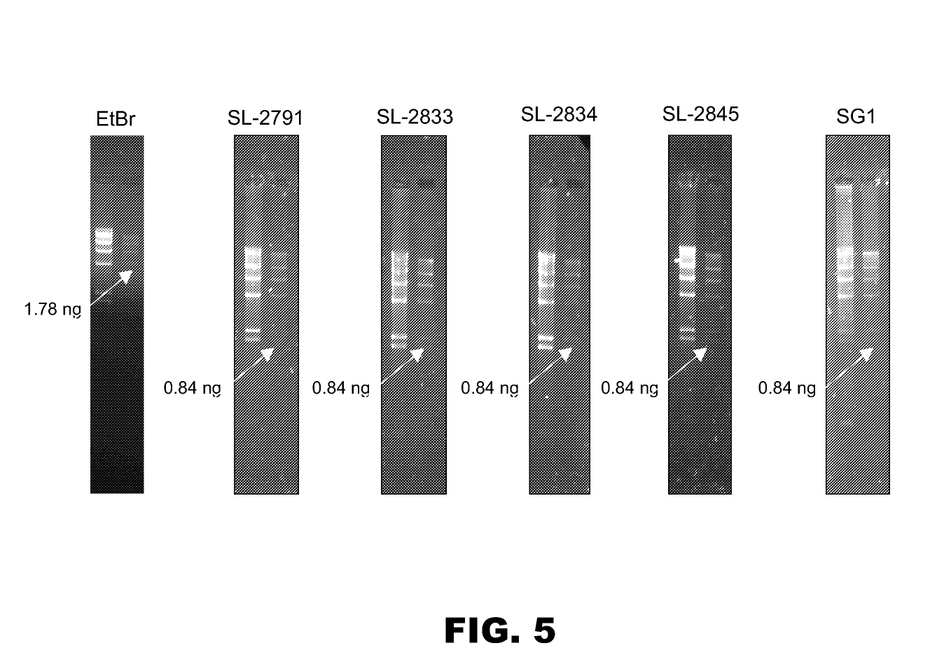

As shown in FIG. 5, all four stains of the invention and SG1 detected less than 1 ng of DNA per lane, whereas EtBr was much less sensitive.

### Example 7

### Digestion of Stained DNA

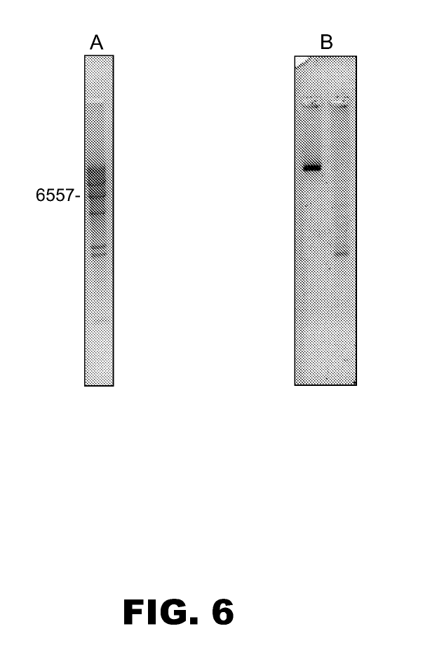

To determine whether the binding between DNA and an unsymmetrical monomethincyanine dye was strong enough to inhibit the digestion of the DNA, HindIII-digested lambda DNA (1 82 g/lane) was resolved on an agarose gel (0.8%), as described above. The DNA was stained either during or after electrophoresis. The band-of-interest (e.g., the 6557 bp band) was excised from the gel using a blue light transilluminator [see FIG. 6A, the DNA was stained with 6 (SL-2791)]. DNA was eluted from the piece of agarose using a spin column (GenElute Agarose Spin Column, Cat. No. 5-6500; Sigma-Aldrich). The collected DNA was digested with an appropriate restriction enzyme, and the reaction products were analyzed on a new agarose gel (1.2%) using compound 9 (SL-2845). The left lane of FIG. 6B presents the undigested DNA and the right lane of FIG. 6B presents the digested products. Thus, DNA stained with an unsymmetrical monomethincyanine dye of the invention may be enzymatically digested.

### Example 8

### Staining of DNA in solution

DNA was stained in solution with compound 9 (SL-2845). Concentrations of DNA ranging from 0 to 10 μg/ml were prepared in 1× TBE solution, pH 8.3 (the DNA was PstI-digested lambda DNA (Cat. No. D1793) as used above). The assay was performed in a 96-well glass-bottom plate, and detection was done on a Laser-Scanner (Fuji FLA-3000) with 532 nm excitation and 580 nm emission filters.

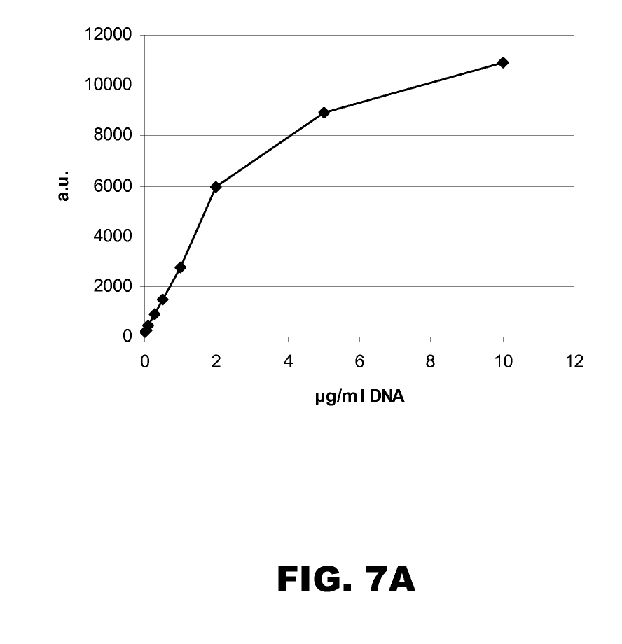

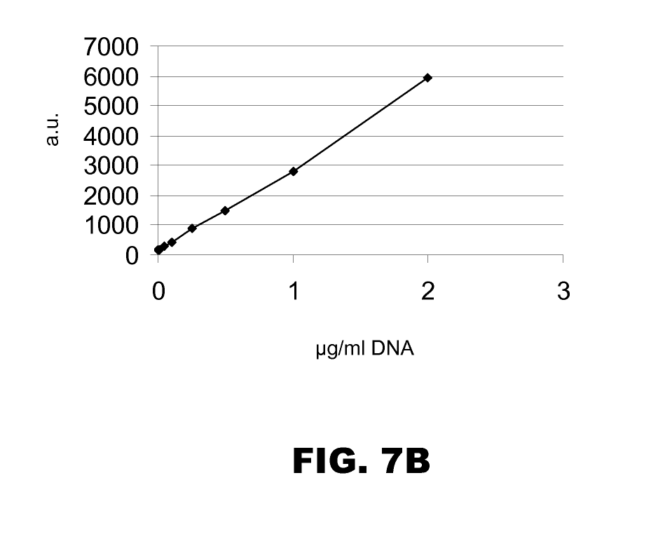

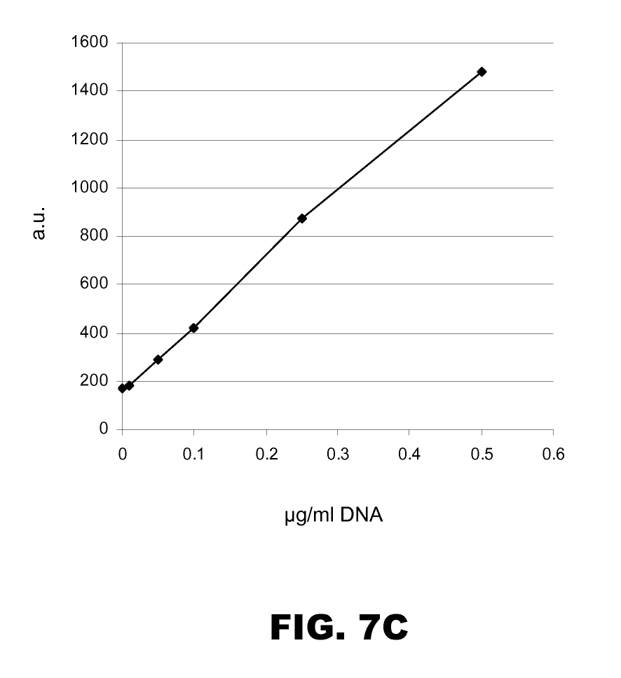

FIG. 7A presents a plot of the fluorescence values as a function of DNA concentration. There was a linear range from 0 to about to 2 μg/ml DNA (FIG. 7B), and quantification was even possible in the low range between 0 and 0.5 μg/ml DNA (FIG. 7C).

The fluorescence spectrum of compound 9 (SL-2845) (at 2 μM) in the presence of 1 μg/ml DNA is presented in FIG. 8. The fluorescence was measured in a cuvette on a Varian Cary Eclipse Fluorescence Spectrophotometer. The excitation spectrum was measured at a fixed emission wavelength of 560 nm, and the emission spectrum was measured at a fixed excitation wavelength of 520 nm. As shown in FIG. 8, the excitation maximum was about 520 nm and the emission maximum was about 565 nm.

What is claimed is:

1. A compound comprising Formula (I): 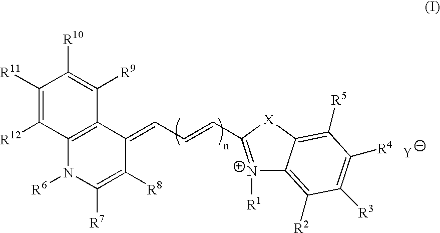
wherein:
R¹ is {—}CH₂(R¹³)ₘOH;
R², R³, R⁴, R⁵, R⁶, R⁸, R⁹, R¹⁰, R¹¹, and R¹² are independently selected from the group consisting of hydrogen, halogen, hydrocarbyl, and substituted hydrocarbyl; provided that any two adjacent substituents may form an aromatic ring or heteroaromatic ring;
R⁷ is a moiety comprising an aromatic ring or a heteroaromatic ring;
R¹³ is selected from the group consisting of hydrocarbyl and substituted hydrocarbyl;
X is a heteroatom;
Y⁻ is a counteranion;
m is an integer from 0 to 10; and
n is an integer from 0 to 5.

2. The compound of claim 1, wherein X is a sulfur atom; R¹ is {—}CH₂(CH₂)ₘOH;
m is from 0 to 5; and n is from 0 to 3.

3. The compound of claim 2, wherein R⁷ is a phenyl ring.

4. The compound of claim 1, comprising Formula (II): 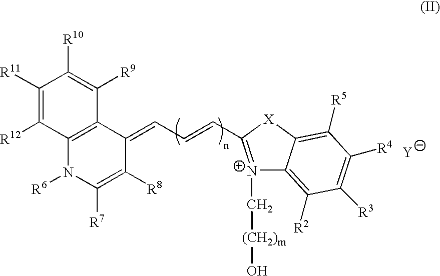
wherein:
R², R³, R⁴, R⁵, R⁶, R⁸, R⁹, R¹⁰, R¹¹, and R¹² are independently selected from the group consisting of hydrogen, halogen, alkyl, alkenyl, and alkoxy; provided that any two adjacent substituents may form an aromatic ring or heteroaromatic ring;
R⁷ is a moiety comprising an aromatic ring or a heteroaromatic ring;
X is selected from the group consisting of a sulfur atom, an oxygen atom, and {—}C(CH₃)₂;
Y⁻ is a counteranion;
m is an integer from 0 to 10; and
n is an integer from 0 to 5.

5. The compound of claim 4, wherein X is a sulfur atom; R⁷ is a phenyl ring; m is from 0 to 5; and n is from 0 to 3.

6. The compound of claim 5, wherein R², R³, R⁴, R⁵, R⁸, R⁹, R¹¹, and R¹² are hydrogen; and R⁶ and R¹⁰ are independently selected from the group consisting of a hydrogen atom and a methyl group.

7. The compound of claim 1, comprising Formula (III): 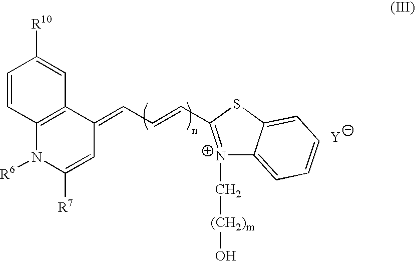
wherein:
R⁶ is selected from the group consisting of a hydrogen atom and a methyl group;
R⁷ is a moiety comprising an aromatic ring or a heteroaromatic ring;
R¹⁰ is selected from the group consisting of a hydrogen atom and a methyl group;
Y⁻ is a counteranion;
m is an integer from 0 to 5; and
n is an integer from 0 to 3.

8. The compound of claim 7, wherein R⁶ is a methyl group, and R⁷ is a phenyl ring.

9. The compound of claim 1, wherein the compound is selected from the group consisting of 6-11:

| | |
|---|---|
| 6 | 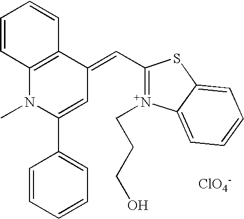 |
| 7 | 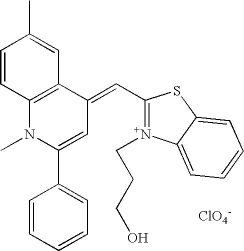 |
| 8 | 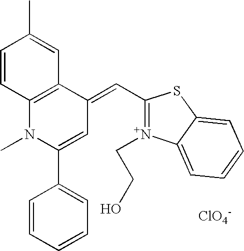 |
| 9 | 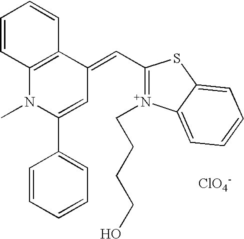 |
| 10 | 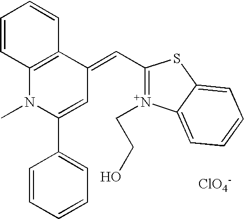 |
| 11 | 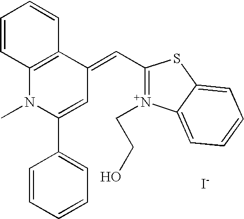 |

10. A complex comprising at least one compound non-covalently bound to a nucleic acid, the compound comprising Formula (I): 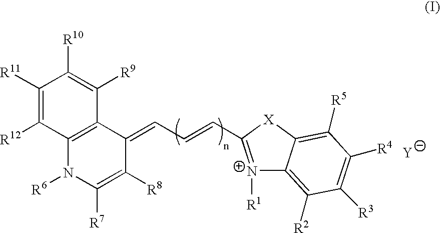
wherein:
R¹ is {—}CH₂(R¹³)ₘOH;
R², R³, R⁴, R⁵, R⁶, R⁸, R⁹, R¹⁰, R¹¹, and R¹² are independently selected from the group consisting of hydrogen, halogen, hydrocarbyl, and substituted hydrocarbyl; provided that any two adjacent substituents may form an aromatic ring or heteroaromatic ring;
R⁷ is a moiety comprising an aromatic ring or a heteroaromatic ring;
R¹³ is selected from the group consisting of hydrocarbyl and substituted hydrocarbyl;
X is a heteroatom;
Y⁻ is a counteranion;
m is an integer from 0 to 10; and
n is an integer from 0 to 5.

11. The complex of claim 10, wherein the compound comprises Formula (II): 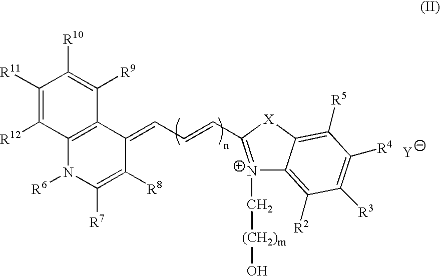
wherein:
R², R³, R⁴, R⁵, R⁶, R⁸, R⁹, R¹⁰, R¹¹, R¹² are independently selected from the group consisting of hydrogen, halogen, alkyl, alkenyl, and alkoxy;
provided that any two adjacent substituents may form an aromatic ring or heteroaromatic ring;
R⁷ is a moiety comprising an aromatic ring or a heteroaromatic ring;
X is selected from the group consisting of a sulfur atom, an oxygen atom, and {—}C(CH₃)₂;
Y⁻ is a counteranion;
m is an integer from 0 to 10; and
n is an integer from 0 to 5.

12. The complex of claim 10, wherein the compound comprises Formula (III): 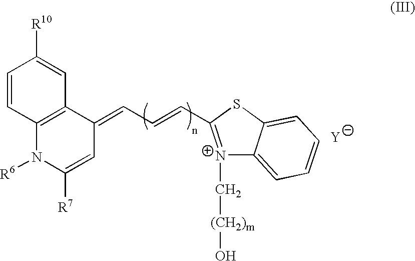
wherein:
R⁶ is selected from the group consisting of a hydrogen atom and a methyl group;
R⁷ is a moiety comprising an aromatic ring or a heteroaromatic ring;
R¹⁰ is selected from the group consisting of a hydrogen atom and a methyl group;
Y⁻ is a counteranion;
m is an integer from 0 to 5; and
n is an integer from 0 to 3.

13. The complex of claim 10, wherein the compound is selected from the group consisting of 6-11:

| | |
|---|---|
| 6 | 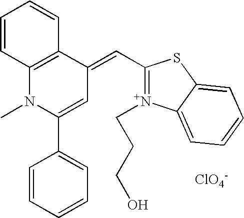 |
| 7 | 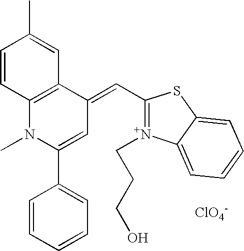 |
| 8 | 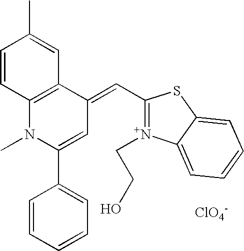 |
| 9 | 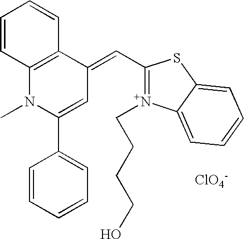 |
| 10 | 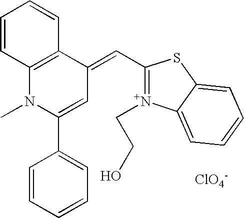 |
| 11 | 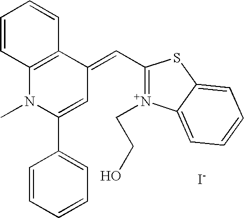 |

14. A method for staining a nucleic acid, the method comprising contacting the nucleic acid with a compound to form at least one non-covalently bound compound-nucleic acid complex that produces a detectable fluorescent signal, the compound comprising Formula (I): 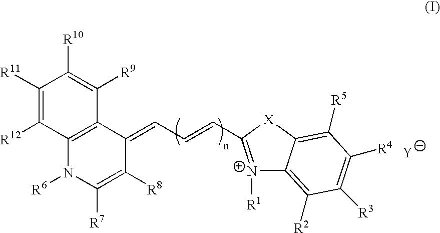
wherein:
R¹ is {—}CH₂(R¹³)ₘOH;
R², R³, R⁴, R⁵, R⁶, R⁸, R⁹, R¹⁰, R¹¹, and R¹² are independently selected from the group consisting of hydrogen, halogen, hydrocarbyl, and substituted hydrocarbyl; provided that any two adjacent substituents may form an aromatic ring or heteroaromatic ring;
R⁷ is a moiety comprising an aromatic ring or a heteroaromatic ring;
R¹³ is selected from the group consisting of hydrocarbyl and substituted hydrocarbyl;
X is a heteroatom;
Y⁻ is a counteranion;
m is an integer from 0 to 10; and
n is an integer from 0 to 5.

15. The method of claim 14, wherein the compound comprises Formula (II): 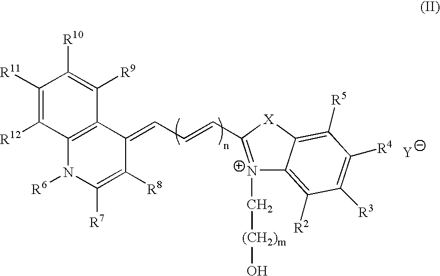
wherein:
R², R³, R⁴, R⁵, R⁶, R⁸, R⁹, R¹⁰, R¹¹, R¹² are independently selected from the group consisting of hydrogen, halogen, alkyl, alkenyl, and alkoxy; provided that any two adjacent substituents may form an aromatic ring or heteroaromatic ring;
R⁷ is a moiety comprising an aromatic ring or a heteroaromatic ring;
X is selected from the group consisting of a sulfur atom, an oxygen atom, and {—C(CH₃)₂;
Y⁻ is a counteranion;
m is an integer from 0 to 10; and
n is an integer from 0 to 5.

16. The method of claim 14, wherein the compound comprises Formula (III): 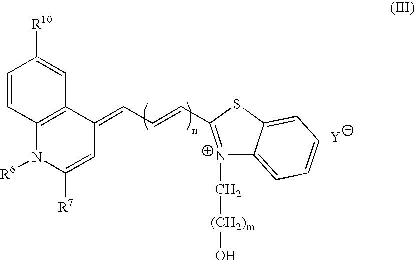
wherein:
R⁶ is selected from the group consisting of a hydrogen atom and a methyl group;
R⁷ is a moiety comprising an aromatic ring or a heteroaromatic ring;
R¹⁰ is selected from the group consisting of a hydrogen atom and a methyl group;
Y⁻ is a counteranion;
m is an integer from 0 to 5; and
n is an integer from 0 to 3.

17. The method of claim 14, wherein the compound is selected from the group consisting of 6-11:

| | |
|---|---|
| 6 | 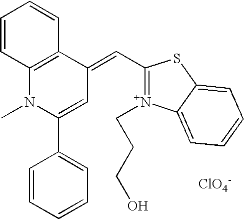 |
| 7 | 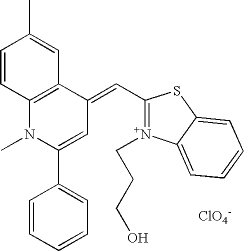 |
| 8 | 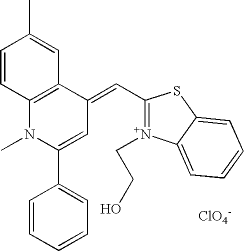 |
| 9 | 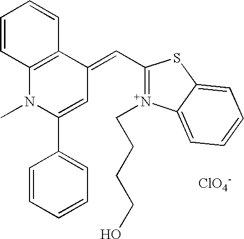 |
| 10 | 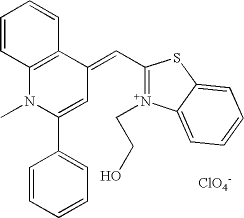 |
| 11 | 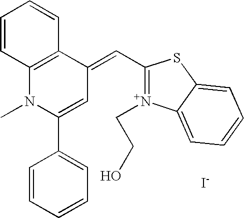 |

18. The method of claim 14, wherein the fluorescent signal is greater upon binding of the compound to a double stranded nucleic acid that upon binding of the compound to a single stranded nucleic acid.

19. The method of claim 14, wherein the compound-nucleic acid complex is on a solid support.

20. The method of claim 19, wherein the solid support is selected from the group consisting of an electrophoretic matrix, a microarray, and a transfer membrane.

21. The method of claim 20, further comprising excising the compound-nucleic acid complex from the electrophoretic matrix, extracting the nucleic acid from the electrophoretic matrix, enzymatically digesting the nucleic acid, and forming new compound-nucleic acid complexes on a second electrophoretic matrix.

22. The method of claim 14, wherein the compound-nucleic acid complex is in an aqueous solution.

23. The method of claim 14, wherein the compound-nucleic acid complex is in a permeabilized cell.

24. The method of claim 14, wherein the compound-nucleic acid complex has an excitation maximum of at least 510 nm.

25. The method of claim 14, wherein the compound-nucleic acid complex fluoresces upon excitation with ultraviolet or blue light.
